(19) United States
(12) Patent Application Publication
(10) Pub. No.: US 2026/0295287 A1
(43) Pub. Date: Oct. 1, 2026

(54) FACE MASK AND A METHOD OF MANUFACTURE OF A FACE MASK

(71) Applicant: I-SMART DEVELOPMENTS LIMITED, Birmingham (GB)

(72) Inventors: Susan Patricia D’ARCY, Birmingham (GB); James Henry WOODHEAD, Birmingham (GB); Jonathan James KNIGHT, Birmingham (GB)

(21) Appl. No.: 19/577,283

(22) Filed: Mar. 24, 2026

(30) Foreign Application Priority Data
Mar. 25, 2025 (GB) 2504331.6
Jul. 2, 2025 (GB) 2510682.4
Oct. 3, 2025 (GB) 2516504.4
Dec. 18, 2025 (GB) 2522139.1

(51) Int. Cl. A61N 5/06; A45D 44/00; A61N 1/04; A61N 7/00

(52) U.S. Cl. CPC A61N 5/06; A45D 44/002; A61N 1/0428; A61N 1/0456; A61N 7/00; A61N 2005/0647; A61N 2005/0652; A61N 2005/0659; A61N 2005/0662

(57) ABSTRACT

The present disclosure relates to a face mask and, more specifically, a face mask for providing therapeutic and/or cosmetic treatment to a user's face. The face mask includes a flexible and stretchable base substrate; a stretchable electronic circuit directly mounted to the base substrate; at least one treatment element in communication with the stretchable electronic circuit; and a flexible and stretchable electrically insulative lamination layer covering the stretchable electronic circuit. The present disclosure also provides a method of manufacturing a face mask for providing therapeutic and/or cosmetic treatment to a user's face.

### CROSS-REFERENCE TO RELATED APPLICATIONS

This application claims priority to GB Patent Application No. 2504331.6 which was filed on 25 Mar. 2025, GB Patent Application No. 2510682.4 which was filed on 2 Jul. 2025, GB Patent Application No. 2516504.4 which was filed on 3 Oct. 2025, and GB Patent Application No. 2522139.1 which was filed on 18 Dec. 2025, the contents of which are incorporated by reference in their entirety herein.

### TECHNICAL FIELD

The present disclosure relates to a face mask and, more specifically, a face mask for providing therapeutic and/or cosmetic treatment to a user's face. In addition, the present disclosure relates to a method of manufacture of a face mask.

### BACKGROUND

Face masks, and other treatment devices, for providing LED therapy or phototherapy are known. These devices are used to apply therapeutic and/or cosmetic treatment to the skin and/or face by, for example, applying different wavelengths of light to the skin to cure diseases or conditions or otherwise improve the skin condition. Other therapeutic and/or cosmetic treatments which may be applied using face masks may include electro muscle stimulation or ultrasound treatment.

These face masks often take the form of a rigid or semi rigid body to which treatment elements are mounted or embedded therein. For example, prior publication KR20200035711A describes a face mask with a 3D shape. Since this mask has a rigid 3D shape, a user whose face does not perfectly match the rigid 3D shape may find the mask ill-fitting and uncomfortable. Since there may be a large variation between users, this necessarily means that a large number of users may find such a mask design ill-fitting.

In addition, since the face mask is rigid or semi-rigid, the face mask is not able to conform closely to the face. Therefore, the prior art face masks may be uncomfortable to wear. Furthermore, since the prior art face masks are not able to conform closely to the face, there will be a variation in the distance between a treatment element of the face mask and the face. This may result in a variation in the efficacy of the face mask across the user's face.

The prior art face mask also have disadvantages associated with manufacturing efficiency. Since they are formed of several layers with specific 3D shapes, they may require manufacturing steps such as moulding and over moulding. These processes may be labour intensive and high cost. Therefore, the manufacturing process may be inefficient.

There is a need for an improved face mask which takes account of at least some of the above issues.

### SUMMARY

There is therefore provided a face mask for providing therapeutic and/or cosmetic treatment to a user's face according to claim 1. The face mask includes: a flexible and stretchable base substrate; a stretchable electronic circuit directly mounted to the base substrate; at least one treatment element in communication with the stretchable electronic circuit; and a flexible and stretchable electrically insulative lamination layer covering the stretchable electronic circuit.

Since the face mask includes the flexible and stretchable base substrate to which the stretchable electronic circuit is directly mounted, it is made of very few components and can have a thin and flexible overall composition. In addition, the stretchable electrically insulative lamination layer covering the stretchable electronic circuit ensures that the face mask can be in direct contact with the skin of the face without further layers.

Since the base substrate, electronic circuit, and electrically insulative lamination layer are all stretchable, the materials can be selected so that they have the same material response. This can allow the face mask to be flexible without introducing strains into the various materials, which could cause delamination of the layers, or other defects. This can allow the face mask to be fully flexible.

The overall effect of these features is a thin and lightweight face mask which can be worn on, and conform to, the skin of the user's face. This can result in a comfortable fit for the user. In addition, since the face mask can conform to the face, there can be a degree of uniformity of the distance between a treatment element of the face mask and the skin, and a reduction in such distance, across the face mask. This can result in a uniformity and optimisation of the efficacy of the face mask across the user's face.

In the present disclosure, stretchable may mean at least as or more stretchable than a polyurethane elastomer and/or a polyolefin elastomer. In addition, and/or alternatively, stretchable may mean the material has an elastic modulus below 30 MPa.

In the present disclosure, flexible may mean at least as or more flexible than polyurethane elastomer and/or a polyolefin elastomer. In addition, and/or alternatively, flexible may mean the material has a stiffness and/or a hardness below 80 Shore A hardness.

The base substrate may include a plurality of substrate layers. In particular, the base substrate may include at least a first substrate layer, second substrate layer, and third substrate layer. The second substrate layer may be provided between the first substrate layer and the third substrate layer. The second substrate layer may therefore be termed the “middle” substrate layer. The middle substrate layer may include an optically reflective surface which may be arranged to reflect emissions from the treatment element towards the skin of the wearer during use. The first substrate layer and the second substrate layer may be formed of any material, for example a thermoplastic polyurethane (TPU).

The electrically insulative lamination layer may include an outer cover, which may be formed of TPU, preferably a transparent TPU. The outer cover may be thermally insulative.

There is also provided a method of manufacturing a face mask for providing therapeutic and/or cosmetic treatment to a user's face according to claim 17. The method includes providing a flexible and stretchable base substrate; mounting a stretchable electronic circuit directly to the base substrate; mounting at least one treatment element in communication with the stretchable electronic circuit; and applying a flexible and stretchable electrically insulative lamination layer over the stretchable electronic circuit.

Since the method involves a simple build-up of flexible layers, it can be formed without complex and labour-intensive processes, such as over moulding. Therefore, this is a more efficient method than those required to manufacture the devices of the prior art.

In embodiments the present disclosure may not be limited to face masks, and instead may be directed more broadly to a treatment device including the technical features as set out herein, with the face mask being a specific example of the treatment device. The treatment device may not be intended only for use on the face but rather on other parts of the body of the user. Therefore, the description herein in relation to face masks may also equally be applied to the treatment device. Therefore, the present disclosure also provides a treatment device according to the following numbered clauses:
Clause 1. A treatment device for providing therapeutic and/or cosmetic treatment to a user's skin, the device including: a flexible and stretchable base substrate; a stretchable electronic circuit directly mounted to the base substrate; at least one treatment element in communication with the stretchable electronic circuit; and a flexible and stretchable electrically insulative lamination layer covering the stretchable electronic circuit.
Clause 2. The treatment device of clause 1, wherein the base substrate includes thermoplastic polyurethane, preferably wherein the thermoplastic polyurethane includes a shore A hardness of 40 to 60, an elastic modulus of 20-40 MPa, and/or a stretchability of 400-500%.
Clause 3. The treatment device of clause 1 or 2, further including a non-stick coating applied to the side of the treatment device including the electrically insulative lamination layer.
Clause 4. The treatment device of any preceding clause, wherein the stretchable electronic circuit includes stretchable conductive ink, preferably wherein the stretchable conductive ink includes a Flexible polymer-chained material mixed with liquid metal alloy, preferably wherein the liquid metal alloy includes silver, carbon, graphite, silver chloride, gold, platinum and/or a conductive trace with a tortuous path.
Clause 5. The treatment device of any preceding clause, wherein the at least one treatment element includes the at least one LED emitter, the at least one laser, the at least one ultrasound emitter, at least one heating/cooling element, at least one TENS element, at least one iontophoresis element and/or the at least one EMS emitters.
Clause 6. The treatment device of clause 5, wherein the LED emitters emit light at a wavelength between 400-1100 nm.
Clause 7. The treatment device of any preceding clause, wherein the at least one treatment element includes a diameter of less than 0.5 mm.
Clause 8. The treatment device of any preceding clause, wherein the at least one treatment element includes at least six treatment elements.
Clause 9. The treatment device of clause 8, wherein the treatment elements are spaced at a spacing of between 5 and 40 mm.
Clause 10. The treatment device of any preceding clause, wherein the electrically insulative lamination layer includes at least one aperture for the at least one treatment element to be located therein, and/or the electrically insulative lamination layer includes a material which transmits an emission from the treatment device.
Clause 11. The treatment device of any preceding clause, wherein the electrically insulative lamination layer includes thermoplastic polyurethane.
Clause 12. The treatment device of any preceding clause, wherein the electrically insulative lamination layer includes the same material as the base substrate.
Clause 13. The treatment device of any preceding clause, further including a treatment element protective layer covering the at least one treatment element, preferably wherein the treatment element protective layer includes a localised silicone drop or polyurethane acrylate (PUA).
Clause 14. The treatment device of clause 13, wherein the treatment element protective layer includes a transparent material.
Clause 15. The treatment device of any preceding clause, further including a strap portion for affixing the treatment device to the user, wherein the strap portion is formed of the same material and/or is unitary with the base substrate, and/or is formed of a different material to the base substrate.
Clause 16. The treatment device of any preceding clause, wherein the treatment device is a face mask, a sleeping bag, a sports sleeve, a Kinaesthetic tape or bandage and/or a patch.
Clause 17. The treatment device of any preceding clause, wherein the treatment device further includes a fabric article to which the stretchable base substrate is mounted.
Clause 18. The treatment device of any preceding clause, wherein the electrically insulative lamination layer includes an outer cover formed of thermoplastic polyurethane (TPU). The outer cover may be thermally insulative.

The disclosure also provides a method of manufacturing a treatment device according to the following numbered clauses:
Clause 19. A method of manufacturing a treatment device for providing therapeutic and/or cosmetic treatment to a user's skin, the method including: providing a flexible and stretchable base substrate; mounting a stretchable electronic circuit directly to the base substrate; mounting at least one treatment element in communication with the stretchable electronic circuit; and applying a flexible and stretchable electrically insulative lamination layer over the stretchable electronic circuit.
Clause 20. The method of clause 19, further including cutting around the treatment device to remove it from excess base substrate and form a shape of the treatment device.
Clause 21. The method of clause 19 or 20, wherein the base substrate is mounted on a release paper layer during manufacture, and the method further includes removing the treatment device from the release paper.
Clause 22. The method of any of clauses 19 to 21, wherein mounting the stretchable electronic circuit directly to the base substrate includes printing the stretchable electronic circuit on the base substrate using a conductive ink.
Clause 23. The method of any of clauses 19 to 22, wherein mounting at least one treatment element in communication with the stretchable electronic circuit includes co-bonding the treatment element to the stretchable electronic circuit.

There is also provided a method of: providing a face mask including a flexible base substrate with distinct chin strap portions; and thermally joining the distinct chin strap portions together to form a unified chin strap. Optionally, the face mask includes TPU and may include, in accordance with the aforementioned face mask, a stretchable electronic circuit directly mounted to the base substrate; at least one treatment element in communication with the stretchable electronic circuit; and a flexible and stretchable electrically insulative lamination layer covering the stretchable electronic circuit.

The present invention has thus far been described in relation to a face mask or treatment device including stretchable materials. That being said, the present invention may also be applicable to other materials, not necessarily stretchable in nature. Therefore, the present disclosure also provides a treatment device according to the following numbered clauses:
Clause 1. A treatment device for providing therapeutic and/or cosmetic treatment to a user's skin, the device including: a flexible base substrate; an electronic circuit directly mounted to the base substrate; at least one treatment element in communication with the electronic circuit; and a flexible electrically insulative lamination layer covering the electronic circuit.
Clause 2. The treatment device of clause 1, wherein the base substrate includes thermoplastic polyurethane, preferably wherein the thermoplastic polyurethane includes a shore A hardness of 40 to 60, an elastic modulus of 20-40 MPa, and/or a stretchability of 400-500%.
Clause 3. The treatment device of clause 1 or 2, further including a non-stick coating applied to the side of the treatment device including the electrically insulative lamination layer.
Clause 4. The treatment device of any preceding clause, wherein the electronic circuit includes conductive ink, preferably wherein the conductive ink includes a Flexible polymer-chained material mixed with liquid metal alloy, preferably wherein the liquid metal alloy includes silver, carbon, graphite, silver chloride, gold, platinum and/or a conductive trace with a tortuous path, preferably wherein the conductive ink is a stretchable conductive ink.
Clause 5. The treatment device of any preceding clause, wherein the at least one treatment element includes the at least one LED emitter, the at least one laser, the at least one ultrasound emitter, at least one heating/cooling element, at least one TENS element, at least one iontophoresis element and/or the at least one EMS emitters.
Clause 6. The treatment device of clause 5, wherein the LED emitters emit light at a wavelength between 400-1100 nm.
Clause 7. The treatment device of any preceding clause, wherein the at least one treatment element includes a diameter of less than 0.5 mm.
Clause 8. The treatment device of any preceding clause, wherein the at least one treatment element includes at least six treatment elements.
Clause 9. The treatment device of clause 8, wherein the treatment elements are spaced at a spacing of between 5 and 40 mm.
Clause 10. The treatment device of any preceding clause, wherein the electrically insulative lamination layer includes at least one aperture for the at least one treatment element to be located therein, and/or the electrically insulative lamination layer includes a material which transmits an emission from the treatment device.
Clause 11. The treatment device of any preceding clause, wherein the electrically insulative lamination layer includes thermoplastic polyurethane.
Clause 12. The treatment device of any preceding clause, wherein the electrically insulative lamination layer includes the same material as the base substrate.
Clause 13. The treatment device of any of clauses 1 to 10, wherein the electrically insulative lamination layer includes a cured liquid silicone rubber.
Clause 14. The treatment device of any of clauses 1 to 10 and 13, wherein the electrically insulative lamination layer includes a smooth surface.
Clause 15. The treatment device of any preceding clause, further including a treatment element protective layer covering the at least one treatment element, preferably wherein the treatment element protective layer includes a localised silicone drop.
Clause 16. The treatment device of clause 15, wherein the treatment element protective layer includes a transparent material.
Clause 17. The treatment device of any preceding clause, further including a strap portion for affixing the treatment device to the user, wherein the strap portion is formed of the same material and/or is unitary with the base substrate, and/or is formed of a different material to the base substrate.
Clause 18. The treatment device of any preceding clause, wherein the treatment device is a face mask, a sleeping bag, a sports sleeve, a Kinaesthetic tape or bandage and/or a patch.
Clause 19. The treatment device of any preceding clause, wherein the treatment device further includes a fabric article to which the base substrate is mounted.
Clause 20. The treatment device of any preceding clause, wherein the base substrate includes a plurality of substrate layers.
Clause 21. The treatment device of any preceding clause, wherein the bas substrate includes a first substrate layer, a second substrate layer, and a third substrate layer, wherein the second substrate layer is provided between the first substrate layer and the second substrate layer.
Clause 22. The treatment device of clause 21, wherein the second substrate layer is formed of a reflective material, preferably an optically reflective material, and optionally wherein the first substrate layer and the second substrate layer are each formed of a thermoplastic polyurethane.
Clause 23. The treatment device of any preceding clause, includes a plurality of bores, preferably wherein each of the plurality of bores extend through each of the base substrate, electronic circuit, and electrically insulative lamination layer, preferably wherein each of the plurality of bores extend through the entirety of the treatment device from one side to the other side, i.e. from the skin-side to the non-skin-side.

The treatment device may be a face mask, and may include any of the features discussed in relation to the face mask and/or treatment device described above, albeit where the materials of each layer may not necessarily being stretchable. For example, each of the base substrate, electronic circuit and electrically insulative lamination layer may be stretchable or non-stretchable. It will be appreciated, that in the case where at least one of the layers is formed of a non-stretchable material, the resulting treatment device may be non-stretchable in nature.

The electrically insulative lamination layer may include an outer cover, which may be formed of TPU, preferably a transparent TPU. The outer cover may be thermally insulative.

The flexible electrically insulative lamination layer may include liquid silicone rubber (LSR). The flexible electrically insulative lamination layer may form the skin-side surface of the treatment device. The LSR includes a smooth surface on the side arranged to face the skin. The use of LSR is beneficial whereby it can be formed using injection moulding techniques and is allowed to flow freely over the electronic circuit and at least one treatment element in order to form a smoot and/or flat surface. This provides improvements to the comfort of the treatment device.

The disclosure also provides a method of manufacturing a treatment device according to the following numbered clauses:
Clause 24. A method of manufacturing a treatment device for providing therapeutic and/or cosmetic treatment to a user's skin, the method including: providing a flexible base substrate; mounting an electronic circuit to the base substrate; mounting at least one treatment element in communication with the electronic circuit; and applying a flexible electrically insulative lamination layer over the electronic circuit.
Clause 25. The method of clause 24, further including cutting around the treatment device to remove it from excess base substrate and form a shape of the treatment device.
Clause 26. The method of clause 24 or 25, wherein the base substrate is mounted on a release paper layer during manufacture, and the method further includes removing the treatment device from the release paper.
Clause 27. The method of any of clauses 24 to 25, wherein mounting electronic circuit directly to the base substrate includes printing the electronic circuit on the base substrate using a conductive ink.
Clause 28. The method of any of clauses 24 to 27, wherein mounting at least one treatment element in communication with the electronic circuit includes co-bonding the treatment element to the electronic circuit.
Clause 29. The method of any of clauses 24 to 28, wherein applying the flexible electrically insulative lamination layer over the electronic circuit includes providing a liquid silicone rubber by injection moulding and subsequently allowing said liquid silicone rubber to cure, preferably wherein the liquid silicone rubber cures with a smooth surface.
Clause 30. The method of any of clauses 24 to 29, further including the step of providing one or more bores extending through each of the base substrate, electronic circuit, and electrically insulative lamination layer.

By way of example only, embodiments according to the present disclosure are now described with reference to, and as shown in, the accompanying drawings.

### BRIEF DESCRIPTION OF THE DRAWINGS

FIG. 1 is a schematic exploded view of a face mask of the present disclosure;

FIG. 2 is a front view of the face mask of FIG. 1 assembled;

FIG. 3 is a side cross-sectional view of a portion of the face mask of FIG. 1 during manufacture;

FIG. 4 is a side cross-sectional view of a portion of the face mask of FIG. 1 according to an additional embodiment;

FIG. 5 is a side cross-sectional view of a portion of the face mask of FIG. 1;

FIG. 6 is a side cross-sectional view of a portion of the face mask of FIG. 1;

FIG. 7 is a side cross-sectional view of a base substrate of the face mask of FIG. 1;

FIG. 8 is a flow diagram of a method of the present disclosure;

FIG. 9a to 9e is a method of manufacture of the face mask; and

FIGS. 10a and 10b are schematics of a further face mask of the present disclosure.

### DETAILED DESCRIPTION

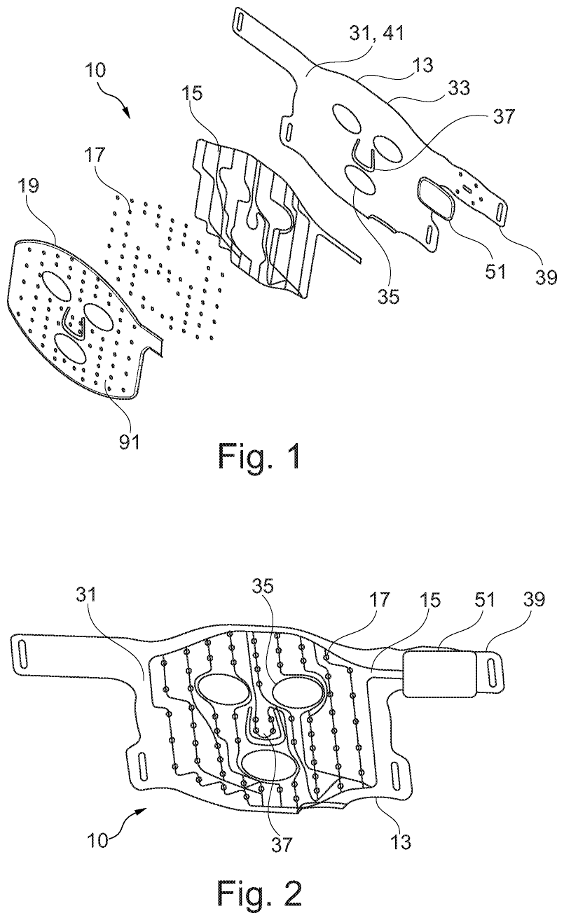

FIG. 1 shows a schematic exploded view of a face mask 10 of the present disclosure. The face mask 10 is for providing therapeutic and/or cosmetic treatment to a user's skin and more particularly, the user's face. Therapeutic treatment refers to the treatment of diseases or disorders by using healing agents or methods. Cosmetic treatment refers to treatment intended to enhance and reshape structures of the body to improve appearance. In the context of the present invention, therapeutic and/or cosmetic treatment includes phototherapy, by, for example, LED emitters, ultrasound therapy, and/or Electro Muscle Stimulation (EMS) treatment for the face, or any skin treatment method that may be incorporated into the face mask 10.

The face mask 10 includes a flexible and stretchable base substrate 13, a stretchable electronic circuit 15, at least one treatment element 17, and a flexible and stretchable electrically insulative lamination layer 19.

The use of stretchable materials may offer certain advantages, but as an alternatively, one or more of the base substrate 13, electronic circuit 15 electrically insulative lamination layer 19 may be formed of any material, not necessarily a stretchable material. For example, at least the electrically insulative lamination layer 19 may be formed of a non-stretchable material. Furthermore, the electrical circuit may be formed of a non-stretchable material. It will be appreciated that forming at least one of the layers from a non-stretchable material would mean that the face mask as a whole would be non-stretchable in nature, albeit one or more of the layers may still be formed from stretchable materials.

FIG. 2 shows the face mask 10 upon assembly, with the electrically insulative lamination layer 19 omitted for clarity. Upon assembly, the stretchable electronic circuit 15 is mounted to the base substrate 13 and the at least one treatment element 17 is in communication with the stretchable electronic circuit 15. Although not shown in FIG. 2, the electrically insulative lamination layer 19 covers the stretchable electronic circuit 15.

The base substrate 13 provides a base upon which the other components of the face mask 10 can be mounted. The base substrate 13 may be made from a variety of different materials. A non-limiting list of the material options includes: Polyimide (PI), Polyester (PET), Polyethylene Naphtholate (PEN), Thermoplastic Polyurethane (TPU), Polycarbonate (PC), Polyethylene (PE), Polypropylene (PP), Polyvinyl Chloride (PVC), Liquid Silicone Rubber (LSR), PU Elastomer, POE (Polyolefin Elastomer), and TPE (Thermoplastic Elastomer)

Of these materials, the preferable material for the base substrate 13 is thermoplastic polyurethane (TPU), for several reasons, including that it is a stretchable material. It is also a material upon which it is suitable to mount the stretchable circuit 15. The mounting process may necessitate the thermal lamination of two substrate layers together, which may require that the material of the base substrate 13 possesses sufficient adhesive strength, i.e., a sufficiently high surface energy. Surface energy is a measure of the attraction between a material's constituent molecules to one another, and to other materials' molecules and thus is correlated with adhesion. In addition, during a curing process of the stretchable circuit 15, elevated temperatures (ideally between 120-140 degrees Celsius, in particular 130 degrees Celsius) may be required. Thus, the material may need to exhibit adequate heat resistance, which is the case with TPU which can withstand temperatures of about 140 degrees Celsius.

In particular, the TPU of the base substrate 13 may be a grade of TPU which includes a shore A hardness of 40 to 60, an elastic modulus of 20-40 MPa, and/or a stretchability of 400-500%. This grade of TPU may provide high transparency, a soft tactile feel, and/or excellent biocompatibility. Overall, this can provide a comfortable wearing experience for the user, and a good fit. If higher environmental sustainability is required, a bio-based TPU may be selected.

Other materials may compromise on stretchability; or lack sufficient heat resistance, necessitating longer curing times; or have low surface energy, requiring special treatment to achieve adhesion, among other drawbacks. Therefore, TPU is the preferred material. However, other materials could also be used, and composite materials may also offer effective performance.

Alternatively, the base substrate 13 could include another flexible and stretchable material such as a fabric. In particular, the base substrate 13 beneficially may include a material to which the stretchable electronic circuit 15 can be mounted and/or attached. Therefore, the base substrate 13 may preferably not include silicone, to which certain stretchable electronic circuits 15 may not be able to be directly mounted easily, due to its relatively low adhesion and surface energy. The base substrate 13 may be opaque, coloured, or alternatively transparent. The base substrate 13 may include a thickness of between 0.025-1.5 mm.

The base substrate 13 is stretchable and flexible, which can allow the face mask 10 to conform to the user's face. Stretchable may mean at least as or more stretchable than a polyurethane elastomer and/or a polyolefin elastomer. In addition, and/or alternatively, stretchable may mean the material has an elastic module below 30 MPa. Flexible may mean at least as or more flexible than polyurethane elastomer and/or a polyolefin elastomer. In addition, and/or alternatively, Flexible may mean the material has a stiffness below 80 Shore A hardness.

The base substrate 13 includes a first side 31 and a second side 33. The first side 31 is the side to which the stretchable electronic circuit 15 is mounted, and the second side 33 is the side which faces away from the user, and may be visible to others, during use. In the illustrated embodiment, the base substrate 13 includes apertures 35, in particular a mouth aperture and two eye apertures. The apertures 35 allow the face mask 10 to be worn as a face mask, since they ensure the face mask 10 does not cover eyes and/or a mouth of the user. In addition, as illustrated, the base substrate 13 includes a nose portion 37, which has the form of a flap. Flap 37 can ensure the face mask 10 can also apply therapeutic and/or cosmetic treatment to the user's nose, which may protrude further than other parts of the face. If, instead of the facemask 10, a more general treatment device is considered which is intended for use on other body parts of the user, it may include a different array of apertures 35 or flaps 37 than illustrated.

As illustrated, the face mask 10, and the base substrate 13 in particular, may further include an attachment element 39 for affixing the face mask 10 to the user, which may take the form of a strap portion 39. In the illustrated embodiment the face mask 10 includes a face portion 41, in which the eye and mouth apertures 35 and nose flap 37 are formed, and the attachment element 39 includes two strap portions extending away from the face portion 41 such that, in use, the two strap portions 39 can wrap around the user's head, extending away from a face/forehead of the user, in order to affix the face mask 10 to the user. Preferably, the attachment element 39 is formed of the same material and/or is unitary with the base substrate 13. Alternatively, the attachment element 39 may be formed of a different material, such as fabric, and affixed to the base substrate 13.

The stretchable electronic circuit 15 is now described in more detail.

The stretchable electronic circuit 15 allows electrical power and signals to be communicated to the at least one treatment element 17. The electronic circuit 15 can communicate power and signals from a power source and/or controller 51. A stretchable circuit is used, rather than a standard circuit of inflexible components, because otherwise stretching or folding of the base substrate 13 may cause a non-stretchable circuit to malfunction. The stretchable circuit 15 may be able to stretch or fold while still functioning correctly. As illustrated, the stretchable circuit 15 is mounted to the first side 31 of the base substrate 13.

The controller 51 is preferably mounted to the strap portion 39 and may include a power source such as a battery, a printed circuit board, and one or more inputs. The power source and/or controller 51 may be mounted to the stretchable electronic circuit 15 and/or connected to the stretchable electronic circuit 15 via a wire using magnetic contacts.

The stretchable electronic circuit 15 preferably includes stretchable conductive ink. The stretchable conductive ink is typically made of flexible polymer-chained material mixed with liquid metal alloy, which can stretch due to the flexible nature of the polymer-chained material. The stretchable conductive ink may include a metal alloy and in particular an alloy including silver, and/or formed from a silver paste. The conductive ink is preferably a printed ink and may have a thickness between 0.003 to 0.03 mm.

Additionally, and/or alternatively, the stretchable electronic circuit 15 may include a conductive trace with a tortuous path. A conductive trace with a tortuous path may be able to stretch because it can extend along an axial direction without stretching the individual wires of the conductive trace, by means of the relative angles of wires within the tortuous path of the trace changing.

The at least one treatment element 17 is now described in more detail.

The at least one treatment element 17 provides therapeutic and/or cosmetic treatment to the user's skin. The at least one treatment element 17 may emit a treatment emission. As illustrated, the at least one treatment element 17 is mounted to the first side 31 of the base substrate 13, such that it is in communication with the stretchable circuit 15.

The at least one treatment element 17 preferably includes at least one LED emitter, in which case the treatment emission may be light. The at least one LED emitter may be configurable to emit light at a wavelength between 400 nm and 1100 nm. In particular, the at least one LED emitter may be configured to emit light with a combination of wavelengths, such as 630 nm and 830 nm, 415 nm and 630 nm, 1072 nm, +/−20 nm (each wavelength may be increased or reduced by 20 nm). Additional wavelengths such as 530 nm, 590 nm, 610 nm, 660 nm, 850 nm, and 940 nm, +/−20 nm may also be used. LED phototherapy may be used to provide skin benefits including: whitening, lightening, de-wrinkling, reduce sensitivity, improve immunity, improve skin barrier, anti-aging, post-care, lifting/firming, acne reduction, wound healing, pigmentation, skin rejuvenation/repair, weight reduction, and/or pain management.

Alternatively, and/or additionally, the at least one treatment element 17 may include at least one ultrasound emitter, a heating/cooling element (such as a Peltier element), and/or at least one EMS emitter depending on what kind of treatments are desired. In these cases, the treatment emission may include ultrasonic radiation, the emission or removal of heat energy, and/or electrical current, respectively.

The at least one treatment element 17 is in communication with the stretchable circuit 15 such that power and signals can be communicated from the controller 51 to the at least one treatment element 17. Therefore, the stretchable circuit 15 may have a layout such that it connects between the controller 51 and all the treatment elements 17.

The at least one treatment element 17 may include a width, diameter, and/or largest dimension of less than 0.5 mm. In particular, the at least one treatment element 17 may be a mini-LED, which is an LED with a width, diameter, and/or largest dimension of less than 0.2 mm or between 0.1 to 0.2 mm. The at least one treatment element 17 may alternatively be a micro-LED, which is an LED with a width, diameter, and/or largest dimension of less than 0.1 mm or between 0.01 to 0.1 mm. Since the treatment elements 17 are typically inflexible, their size can affect the overall flexibility of the face mask 10. Therefore, the use of at least one treatment element 17 of a small size can improve the flexibility of the face mask 10.

The LED, mini-LED, and/or micro-LED may have any shape including circular or square,

The at least one treatment element 17 may include at least six, at least eight, or at least ten treatment elements. In addition, and/or alternatively, the treatment elements 17 may be spaced at a spacing of between 5 and 40 mm, 5 and 30 mm, or between 8 and 20 mm. These parameters may ensure that the face mask 10 has a dense enough distribution, and large enough number of treatment elements 17, to ensure that sufficient efficacy is achieved. At the same time, too dense a distribution, and/or too large a number of treatment elements 17 may reduce the overall flexibility of the face mask 10 so that its usefulness is reduced. These parameters give an optimal compromise between these two considerations.

The flexible and stretchable electrically insulative lamination layer 19 is now described in more detail.

The flexible and stretchable electrically insulative lamination layer 19 provides a protective cover so that the face mask 10 can be worn without the stretchable circuit 15 causing damage and/or discomfort to the user. The electrically insulative lamination layer 19 is applied, preferably by hot-pressing the electrically insulative lamination layer 19, over the stretchable circuit 15 on the opposite side of the stretchable circuit 15 to the base substrate 13. The electrically insulative lamination layer 19 may be applied as a layer to the first side 31 of the base substrate 13 after the stretchable circuit 15 is mounted to the base substrate 13. The electrically insulative lamination layer 19 is flexible and stretchable so that it can deform with the other layers of the face mask 10.

The electrically insulative lamination layer 19 may be formed of TPU and/or may be formed of the same material as the base substrate 13. This can ensure that the electrically insulative lamination layer 19 has the desired material properties. In addition, TPU is an example of an electrically insulative material, and therefore suitable as the electrically insulative lamination layer 19. The electrically insulative lamination layer 19 may include a thickness of between 0.05 to 1.0 mm.

The electrically insulative lamination layer 19, as illustrated, may include at least one aperture 91 for the at least one treatment element 17 to be located therein. This can allow a material to be used for the electrically insulative lamination layer 19 which would otherwise reduce the efficacy of the at least one treatment element 17. For example, if the at least one treatment element 17 includes at least one LED, the electrically insulative lamination layer 19 including a material which blocks light may reduce the efficacy of the at least one treatment element 17 if the electrically insulative lamination layer 19 did not include at least one aperture 91 for the at least one treatment element 17 to be located therein. TPU, which is a suitable material for the electrically insulative lamination layer 19 as explained above, does not have an optimum transparency, and therefore would reduce the efficacy of LEDs if not for the feature of the at least one aperture 91. Therefore, the presence of the at least one aperture 91 allows a material to be used for the electrically insulative lamination layer 19 which may otherwise reduce the efficacy of the at least one treatment element 17.

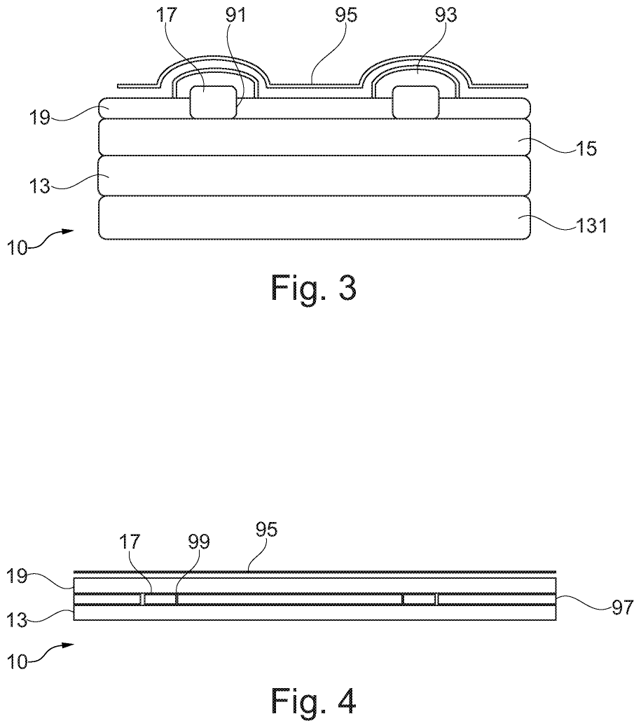

FIG. 3 shows a cross section of a portion of the face mask 10 in which the feature of the at least one aperture 91 is seen more clearly. As can be seen, the or each treatment element 17 extends through the or one of the aperture(s) 91. Therefore, the treatment emission from the treatment element 17 is not blocked by the electrically insulative lamination layer 19 and is able to reach the user with maximum efficacy.

If the electrically insulative lamination layer 19 includes a material which does not reduce the efficacy of the at least one treatment element 17, then the at least one aperture 91 may be omitted. In other words, if the electrically insulative lamination layer 19 transmits the treatment emission of the at least one treatment element 17, then the at least one aperture 91 may be omitted. For example, if the at least one treatment element 17 includes an ultrasound emitter, and the electrically insulative lamination layer 19 is a good transmitter of ultrasound waves, then the at least one aperture 91 may be omitted, as it does not affect the treatment. Additionally, if the electrically insulative lamination layer 19 is transparent, and the treatment emission of the at least one treatment element 17 is light, then the at least one aperture 91 may be omitted. Indeed, if the electrically insulative lamination layer 19 includes a material which does not reduce the efficacy of the at least one treatment element 17, then it may be beneficial to omit the at least one aperture 91 to reduce complexity of the design.

As shown in FIG. 3, the face mask may further include a treatment element protective layer covering the at least one treatment element 17 and at least one aperture 91, such that the at least one treatment element 17 is sealed in the at least one aperture 91. The treatment element protective layer 93 may protect the at least one treatment element 17 from the user's skin, or another environment which could damage the at least one treatment element 17. Preferably the treatment element protective layer 93 includes a material which does not reduce the efficacy of the at least one treatment element 17. If the at least one treatment element 17 includes an LED, then the treatment element protective layer 93 preferably includes a material which is highly transparent, to allow a high transmission of light. Therefore, the treatment element protective layer 93 may include a localised silicone drop, since silicone is highly transparent. Alternatively, the treatment element protective layer 93 may include Polyurethane Acrylate (PUA) in the form of localised PUA drops provided over each treatment element 17. If the at least one treatment element 17 includes a different type of emitter, than the treatment element protective layer 93 may include a material which has a high transmission for that type of emission.

Although in the case of FIG. 3 the treatment element protective layer 93 may sit proud of the electrically insulative lamination layer 19, in the case where a smooth surface may be required, the treatment element protective layer 93 may be flush with the electrically insulative layer 19. In the specific example of a localised silicone or PUA drop, each drop may be sized to fill a respective aperture 91 such that the silicone or PUA does not extend above the top of the aperture 91.

Although not shown in FIG. 3, the electrically insulative lamination layer 19 may further include an outer cover, which may be provided to cover the at least one treatment element 17 and the treatment element protective layer 93. The outer cover may therefore extend over the electrically insulative lamination layer 19 and be provided on the side of the face mask opposite to the base substrate. The outer cover of the electrically insulative lamination layer 19 may be formed of TPU, preferably a transparent TPU in view of the fact that the outer cover may extend over the at least one treatment element. The outer cover may be formed of thermally insulative materials such that the surface of the wearer's skin is insulated from any heat that may be generated by the at least one treatment element 17.

In the arrangement above whereby the treatment element protective layer 93 is flush with the electrically insulative layer 19, the outer cover of the electrically insulative layer may extend over the treatment element protective layer 93 to provide a smooth outer surface.

The treatment element protective layer 93 may include a material which is not suitable as the electrically insulative lamination layer 19. Since the treatment element protective layer 93 is only applied at small, localised locations, it does not significantly affect the overall flexibility of the face mask 10 and therefore can be a material which is substantially non-flexible, or is rigid or semi-rigid, and would not be suitable for the electrically insulative lamination layer 19, which covers a larger area and thus is flexible.

This arrangement using the aperture 91 and the treatment element protective layer 93 allows an optimal material to be used as the electrically insulative lamination layer 19 (such as TPU), even if it would reduce the efficacy of the at least one treatment element 17, and an optimal material to be used as the treatment element protective layer 93, even if it would not be suitable as the electrically insulative lamination layer 19 (such as silicone).

FIG. 3 also illustrates a non-stick coating 95 which the face mask 10 may include. The non-stick coating may be referred to as a feel-good oil and is a functional coating applied to material surfaces, typically composed of low-surface-energy substances (such as silicone, fluoropolymers, or special organic compounds). By forming an ultra-thin film, the feel-good oil imparts a soft and smooth tactile feel to the material while providing properties such as anti-stick, anti-fouling, and wear resistance. The non-stick coating 95 can prevent the material of the face mask 10 sticking to itself, which can allow the face mask 10 to be folded when not in use. In addition, the non-stick coating 95 can provide a pleasant feel and finish to the face mask 10 so that it is comfortable to be worn by the user. The non-stick coating 95 preferably includes an oil base and may be selected from the following non-limiting list: Silicone-Based Oil, Polyurethane (PU)-Based Oil, Fluorocarbon-Based Oil, Acrylic-Based Oil, Nano-Coating Oil, UV-Resistant Oil, Eco-Friendly Water-Based Oil; based on the desired material properties.

The non-stick coating 95 is preferably applied to the side of the face mask 10 including the electrically insulative lamination layer 19 and may be applied to the electrically insulative lamination layer 19 and the treatment element protective layer 93 if present. In addition, the non-stick coating 95 can be applied to the second side 33 of the base substrate 13 so the entire face mask 10 is coated.

FIG. 4 shows an additional and/or alternative structure of the face mask 10. The face mask 10 of FIG. 4 also includes the flexible and stretchable base substrate 13; the stretchable electronic circuit (not shown) directly mounted to the base substrate 13, the at least one treatment element 17 in communication with the stretchable electronic circuit; and the flexible and stretchable electrically insulative lamination layer 19 covering the stretchable electronic circuit. There may be additional layers between the flexible and stretchable base substrate 13, the stretchable electronic circuit, and the flexible and stretchable electrically insulative lamination layer 19. The face mask 10 may further include the non-stick coating 95.

In the example of FIG. 4, the face mask 10 includes an adhesive layer 97. The flexible and stretchable electrically insulative lamination layer 19 is mounted to the stretchable electronic circuit, and/or flexible and stretchable base substrate 13 via and/or using the adhesive layer 97. The adhesive layer 97 allows the electrically insulative lamination layer 19 to be mounted to the other components of the mask 10 using an alternate and/or additional method to hot pressing.

The adhesive layer 97 is preferably an Optically Clear Adhesive (OCA). OCA may be a transparent, solid adhesive film typically used for bonding transparent components, such as in electronic displays. OCA may include a high transparency and durability and may bond materials together without forming bubbles. OCA may be formed of a silicone or acrylic base. The OCA may be highly transparent and may include a light transmittance greater than 90%.

Since the OCA is transparent, the flexible and stretchable base substrate 13 and flexible and stretchable electrically insulative lamination layer 19 may be formed from transparent materials such that the face mask 10 as a whole is transparent. The face mask 10 being transparent may allow an enhanced user experience as it may provide improved peripheral vision for the user during use.

The adhesive layer 97 preferably includes apertures 99. The apertures 99 are arranged for the at least one treatment element 17 to be located therein. As shown in FIG. 4, this arrangement may allow for a symmetrical sandwich structure around the at least one treatment element 17 with the flexible and stretchable base substrate 13 and the stretchable electrically insulative lamination layer 19 as continuous sheets on either side of the at least one treatment element 17. Since the stretchable base substrate 13 and the stretchable electrically insulative lamination layer 19 are continuous sheets without apertures, they may be selected to have similar and/or the same material properties. Therefore, the face mask 10 may stretch and/or flex as a single sheet without introducing undesirable stresses into the materials. This may therefore improve the durability of the face mask 10.

In addition, the adhesive layer 97 may have a thickness equal or similar to the thickness of the at least one treatment element 17. This may allow the face mask 10 to have a smooth surface without protrusions despite the presence of the at least one treatment element 17. This may provide an enhanced user experience and may also improve the durability of the face mask 10.

FIG. 5 depicts a further arrangement of the face mask 10 similar to that described in relation to FIG. 4, wherein a plurality of bores 101 are provided. Each of the bores 101 extend through the layers of the face mask 10, such that they may extend from the non-skin side to the skin side of the face mask. The plurality of bores 101 may be evenly distributed across the face mask 10 and are arranged to allow air to flow to the skin surface when the face mask 10 is in use, and thus improves the breathability of the face mask 10. It will be appreciated that the plurality of bores 101 in this context are intended to refer to bores other than those that would necessarily be present to accommodate the eyes, noes and mouth of the user.

Each of the one or more bores 101 may be formed within each layer of the face mask 10 separately and then aligned during the assembly of the face mask 10. Alternatively, each of the one or more bores 101 may be formed once the layers of the face mask 10 have been assembled together.

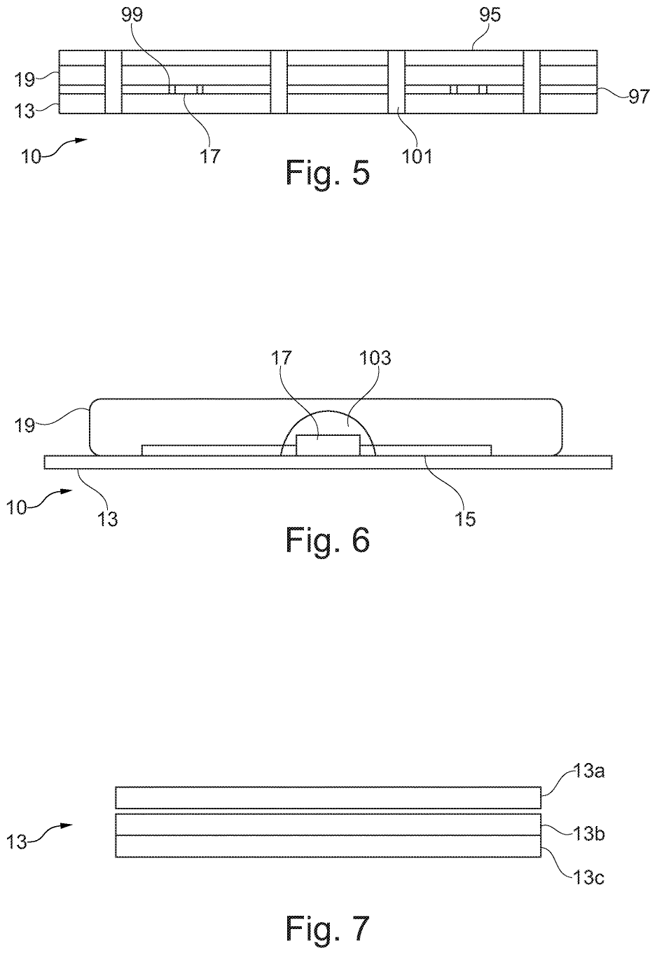

FIG. 6 depicts a lay-up of the face mask 10 including an alternative arrangement of the electrically insulative layer 19. As will be appreciate from FIG. 3, the use of a treatment element protective layer 93 to provide protection to the treatment element 17 may result in a protrusion extending from the surface of the face mask 10. Said protrusion may extend from the skin-side of the face mask, and so may result in an undesirable feel to the surface of the face mask when in use.

In light of this, the electrically insulative layer 19 may include a liquid silicone rubber (LSR) instead of the TPU described in relation to FIG. 3 above. In particular, the LSR may be provided on the surface of the base substrate 13 or electronic circuit 15 and may include a smoot and/or flat surface on the side of the face mask 10 arranged to face the skin of the user. This results in a face mask 10 which is more comfortable for the user to wear due to the smooth surface, as opposed to the protrusions depicted in FIG. 3 above. All other features of the face mask 10 depicted in FIG. 6 may be as described in relation to FIG. 3 above. It will be appreciated that LSR is typically considered a non-stretchable material, and hence when used in combination with the stretchable materials described in relation to the face mask 10 depicted in FIG. 3 above, the resulting face mask may not, therefore, be stretchable in nature. It will be appreciated, therefore, that the remaining layers of the face mask 10 may also optionally include non-stretchable materials or alternatively, they may still include stretchable materials as discussed above. In particular, the base substrate may be formed of a non-stretchable material and the electronic circuit may include a non-stretchable conductive ink.

The LSR of the electrically insulative layer 19 may extend over the at least one treatment element such that the at least one treatment element is completely encased within the LSR.

The at least one treatment element 17 may be fixed to the surface of the electrical circuit and/or the base substrate 13 using an adhesive 103. Moreover, the at least one treatment element 17 may be provided within a recess (not shown) in the surface of the electrical circuit 15 and/or the base substrate 13. The use of a recess to house the at least one treatment element 17 in combination with an adhesive 103 reinforces the position of the at least one treatment element 17 and prevents any undesired movement of the at least one treatment element 17 within the face mask 10.

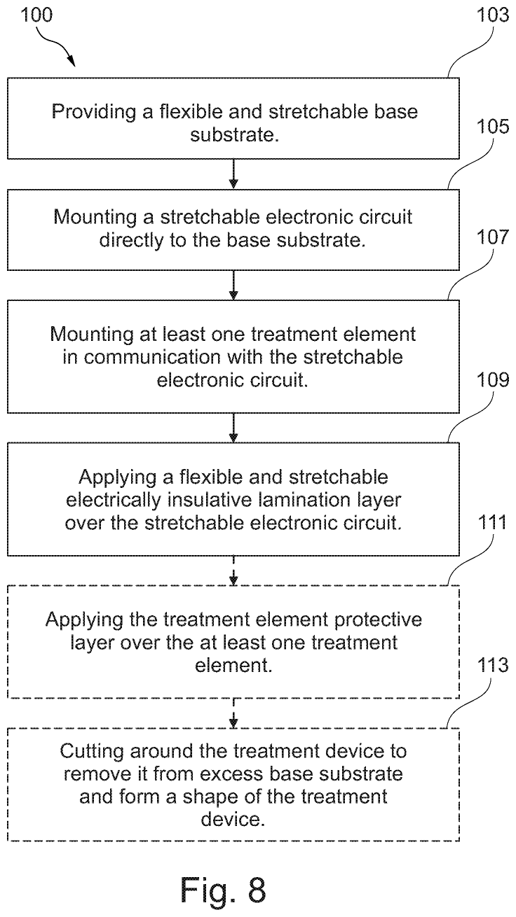

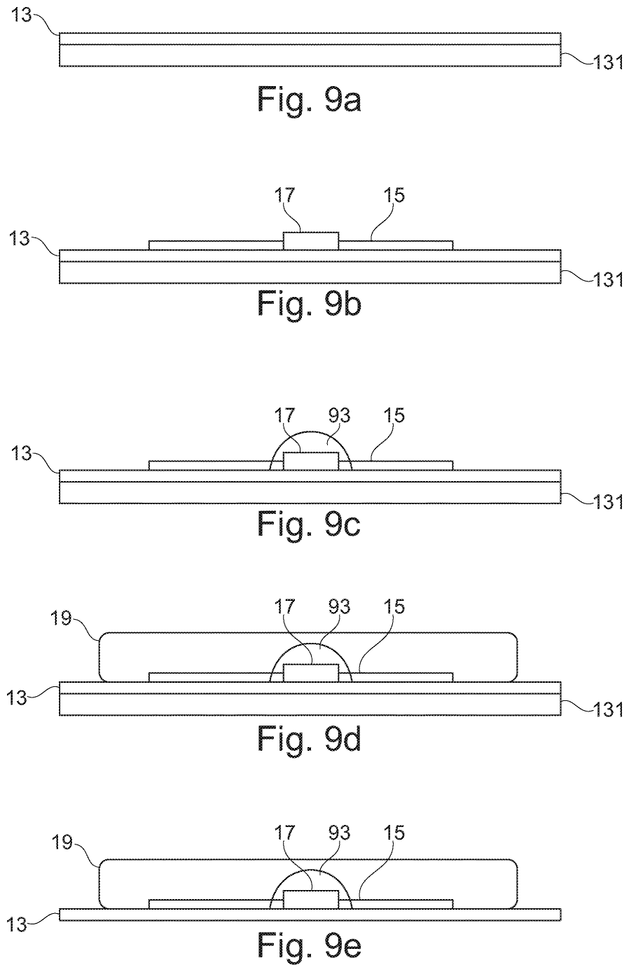

The LSR can be formed using moulding techniques, preferably high-pressure injection moulding, and is freely flowable under gravity. As such, during the manufacturing process (discussed in more detail below in relation to FIGS. 8 and 9a to 9e), with the appropriate mould the LSR can fill an area and settle such that upon solidifying it may include a flat upper surface.

In the embodiment of FIG. 6, the treatment element protective layer 93 may include a silicone drop as discussed above. Additionally, an adhesive drop may be applied to the electrical circuit 15 which the at least one treatment element 17 may be mounted to. The adhesive drop can provide reinforcement and prevent movement of the at least one treatment element 17. The treatment element protective layer 93 in the form of a silicone drop may then be applied to the at least one treatment element 17.

The electrical circuit 15 may be as described in relation to FIG. 3 above. In particular, the electronic circuit 15 preferably includes a conductive ink, which may be a stretchable conductive ink or a non-stretchable conductive ink. The conductive ink is typically made of flexible polymer-chained material mixed with liquid metal alloy, which can stretch due to the flexible nature of the polymer-chained material. The conductive ink may include a metal alloy and in particular an alloy including silver, and/or formed from a silver paste. The conductive ink is preferably a printed ink and may have a thickness between 0.003 to 0.03 mm.

The face mask 10 may include the non-stick coating 95 as described in relation to FIGS. 3 and/or 4 above.

FIG. 7 shows a detailed view of the base substrate 13 used in the face mask 10 which may be provided in combination with any of the face masks 10 depicted in FIG. 3, 4 or 5.

The base substrate 13 include a plurality of substrate layers 13a, 13b, 13c, which together may form the base substrate 13. The base substrate 13 includes a first substrate layer 13a, a second substrate layer 13b, and a third substrate layer 13c. It will be appreciated that additional layers may also be provided.

In the present arrangement, the second substrate layer 13b may be provided between the first substrate layer 13a and the third substrate layer 13c. The first substrate layer 13a and the third substrate layer 13c is formed of any material, such as TPU, and the second substrate layer 13b is formed of a reflective material, preferably an optically reflective material.

The reflective material in the second substrate layer 13b is arranged to reflect any emissions from the at least one treatment element 17 towards the skin-side of the face mask. For example, in the case of the at least one treatment element 17 being a light source, specifically an LED, the optically reflective material of the base substrate 13 is configured to reflect the light from the at least one treatment element 17 towards the skin-side surface of the face mask 10. The use of an optical reflective material within the base substrate 13 is therefore beneficial as it prevents any emissions from the at least one treatment element from “leaking” through the base substrate 13 on the non-skin side of the face mask 10, and thus results in more improved and efficient treatment.

Alternatively, and/or additionally to face masks, the present disclosure may be applied to a general treatment device, which is not intended only for use on the face but rather on other parts of the body of the user as well. For example, the treatment device may include a different device, such as an eye patch, an Acne patch, a dermatitis patch, a neck patch, or a device for treating any part of the body, wherein the device is for applying treatment to the user's skin. In addition, the treatment device could be incorporated into other objects, such as articles including sleeping bags, sports sleeves, Kinaesthetic tape or bandages and/or patches. In such a use, the base substrate 13 may be mounted onto a fabric surface of the article. In this case, the second side 33 of the base substrate 13 would be mounted, attached and/or joined to the article, and this could be accomplished by hot-pressing. The treatment device could also be incorporated into a medical, fabric, face mask, as a chin only patch, and not the entire mask. For example, the portion of the medical, fabric, face mask located under the chin in use may include the treatment device.

It will be appreciated that a general treatment device may include any of the features described in relation to the face mask 10.

A method 100 of manufacturing the face mask 10 is now described, with reference to FIG. 8, including a substrate providing step 103, a circuit mounting step 105, a treatment element mounting step 107, an electrically insulative lamination layer application step 109, an optional treatment element protective layer application step 111, and an optional cutting step 113.

At the substrate providing step 103, the flexible and stretchable base substrate 13 is provided. The base substrate 13 may be provided and/or mounted on a release paper layer 131 (shown in FIG. 3 and FIGS. 9a to 9e) during manufacture. The release paper layer 131 restricts the stretchable base substrate 13 from distortion during manufacture, to ensure manufacturing accuracy, and also provides protection in production.

At the circuit mounting step 105, the stretchable electronic circuit 15 is mounted directly to the base substrate 13. Step 105 preferably includes printing the stretchable electronic circuit 15 onto the base substrate 13 using a conductive ink. Such printing may include ink silk printing.

At the treatment element mounting step 107, the at least one treatment element 17 is mounted in communication with the stretchable electronic circuit 15. Preferably, the at least one treatment element 17 is mounted to the base substrate 13 itself and/or to the circuit 15. The treatment element mounting step 107 preferably includes co-bonding the treatment element 17 to the stretchable electronic circuit 15. Co-bonding refers to the process of placing at least the one treatment element 17, such as at least one mini-LED, directly onto a corresponding pad position(s) on the stretchable circuit 15, before the stretchable circuit 15 is cured. Subsequently, both the stretchable circuit 15 and the treatment elements 17 may be heated together (for example at approximately 140 degrees Celsius). Once the stretchable circuit 15 is cured, it forms a reliable connection with the at least one treatment element 17.

Alternatively, the treatment element mounting step 107 could include using surface mounting technology (SMT), or low-temperature SMT to mount the treatment element 17 to the stretchable electronic circuit 15. For the present method, due to the limited temperature resistance of the thin film substrates used, to which the components are mounted, two key requirements are preferably satisfied by the treatment element mounting process: 1) a low temperature below 140 degrees Celsius; and 2) good soldering strength. Therefore, co-bonding is a particularly preferably process because it satisfies these two requirements. However, other mounting processes may also be implemented.

At the electrically insulative lamination layer application step 109, the flexible and stretchable electrically insulative lamination layer 19 is applied over the stretchable electronic circuit 15. Step 109 may be carried out by hot-pressing the electrically insulative lamination layer application step 109 to the base substate 13 and/or the stretchable electronic circuit 15 so that they bond.

The electrically insulative lamination layer application step 109 in practice may preferably be carried out before or simultaneously to the treatment element mounting step 107. This is especially possible if the at least one aperture 91 of the electrically insulative lamination layer 19 is present. If so, treatment element mounting step 107 may include inserting the at least one treatment element 17 through the at least one aperture 91 to mount the at least one treatment element 17 in communication with the stretchable electronic circuit 15.

An alternative process for the electrically insulative lamination layer application step 109 is depicted in FIGS. 9a to 9e. In particular, the process steps 103, 105, 107, 111, 113 are all as described already. However, once the electrical circuit 15, e.g. the conductive ink 15, and the at least one treatment element 17 have been applied to the base substrate 13, and the protection cover 93 has been provided, the liquid silicone rubber is provided using moulding techniques, preferably high-pressure injection moulding. The method then includes a step of allowing the liquid silicone rubber to cure such that it forms a smooth upper surface, wherein this upper surface is arranged to be the side of the face mask 10 facing the skin of the use, i.e. the skin-side of the face mask 10. Once the liquid silicone rubber 19 has cured, the release paper 131 can be removed. The release paper 131 is necessary up to this step in order to provide sufficient rigidity to the face mask 10 to allow the injection of the liquid silicone rubber.

In the embodiment of FIG. 8, the electrically insulative lamination layer application step 109 may include applying the adhesive layer 97 over the stretchable electronic circuit 15, and the electrically insulative lamination layer 19 on top of the adhesive layer 97. The method may include curing the adhesive layer 97 such that it forms a bond between the stretchable electronic circuit 15 and/or base substrate 13 and the electrically insulative lamination layer 19. The curing process may include UV light, heat and/or pressure.

The electrically insulative lamination layer application step 109 may include cutting the apertures 99 in the adhesive layer 97 prior to applying the adhesive layer 97 over the stretchable electronic circuit 15. The apertures 99 may be cut into the adhesive layer 97 based on the positions of the at least one treatment element 17.

The method 100 may further include the treatment element protective layer application step 111 of applying the treatment element protective layer 93 over the at least one treatment element 17 and/or at least one aperture 91. This may thereby ensure the at least one treatment element 17 is sealed in the at least one aperture 91 from the environment and therefore protected as explained above. The treatment element protective layer application step 111 may include adding a localised silicone drop, or other protective material, on each of the at least one treatment element 17 and/or at least one aperture 91 and allowing the localised protective material to dry and/or cure.

The method 100 may further include cutting step 113 of forming the face mask 10 from the base substrate 13 by cutting the shape of the face mask 10 to remove it from excess base substrate 13. This may thereby form a face mask shape, as shown in FIG. 1. The cutting may include cutting the apertures 35 and the flap 37 simultaneously or separately. The cutting may include laser cutting.

In addition, the method 100 may further include the step of removing the face mask 10 from the release paper layer 131.

The method 100 may further include the step of assembling the controller 51 to the base substrate 13. The controller 51 may be assembled via a magnetic connection, for example.

If the face mask 10 is designed to include other materials, then the method 100 may further include the step of heat transferring the base substrate 13, such as the TPU base substrate 13 with the stretchable electronic circuit 15 and other components, onto a fabric.

As explained above, the present disclosure is not limited to face masks and may also be applied to more general treatment devices designed to apply treatment to other parts of the body. Therefore, method 100 could be used to manufacture a treatment device as well as the face mask 10.

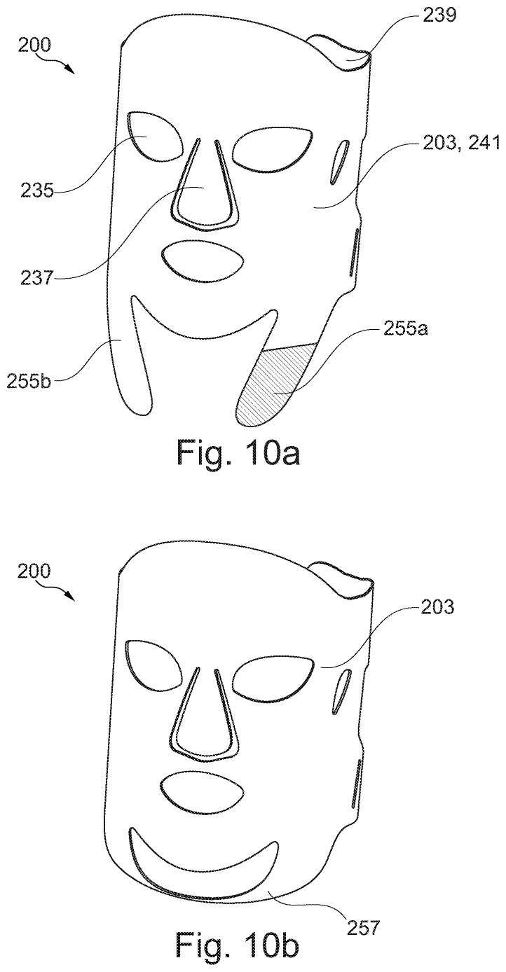

As illustrated in FIGS. 10a and 10b, there is also provided a related invention of a further method of manufacturing a face mask 200.

The method includes providing a face mask 200 including a base substrate 203, which may include TPU. As illustrated, the face mask 200 may include a face portion 241, in which eye and mouth apertures 235 and a nose flap 237 are formed, and an attachment element 239 including a strap portion extending away from the face portion 241 such that, in use, the strap portion 239 wraps around the user's head, extending away from a face/forehead of the user, in order to affix the face mask 200 to the user. Preferably, the attachment element 239 is formed of the same material and/or is unitary with the base substrate 203.

In addition, the face mask 200 includes distinct chin strap portions, namely a first chin strap portion 255a and a second chin strap portion 255b.

The method further includes thermally joining the distinct chin strap portions 255a, 255b together to form a unified chin strap 257, as shown in FIG. 10b. The thermal joining of the distinct chin strap portions may include hot-pressing the chin strap portions 255a, 255b together so that they form a unified chin strap 257.

Preferably, the face mask 200 includes the same features as the face mask 10 as described above, such that the description of face mask 10 equally applies to face mask 200. Hence face mask 200 may include a stretchable electronic circuit directly mounted to the base substrate; at least one treatment element in communication with the stretchable electronic circuit; and/or a flexible and stretchable electrically insulative lamination layer covering the stretchable electronic circuit.

1. A face mask for providing therapeutic and/or cosmetic treatment to a user's face, the face mask comprising:
a flexible and stretchable base substrate;
a stretchable electronic circuit directly mounted to the base substrate;
at least one treatment element in communication with the stretchable electronic circuit; and
a flexible and stretchable electrically insulative lamination layer covering the stretchable electronic circuit.

2. The face mask of claim 1, wherein the base substrate comprises thermoplastic polyurethane, preferably wherein the thermoplastic polyurethane comprises a shore A hardness of 40 to 60, an elastic modulus of 20-40 MPa, and/or a stretchability of 400-500%.

3. The face mask of claim 1, further comprising a non-stick coating applied to the side of the face mask comprising the electrically insulative lamination layer.

4. The face mask of claim 1, wherein the stretchable electronic circuit comprises stretchable conductive ink, preferably wherein the stretchable conductive ink comprises a Flexible polymer-chained material mixed with liquid metal alloy, preferably wherein the liquid metal alloy comprises silver, carbon, graphite, silver chloride, gold, platinum and/or a conductive trace with a tortuous path.

5. The face mask of claim 1, wherein the at least one treatment element comprises the at least one LED emitter, the at least one laser, the at least one ultrasound emitter, at least one heating/cooling element, at least one TENS element, at least one iontophoresis element and/or the at least one EMS emitters.

6. The face mask of claim 5, wherein the LED emitters emit light at a wavelength between 400-1100 nm.

7. The face mask of claim 1, wherein the at least one treatment element comprises a diameter of less than 0.5 mm.

8. The face mask of claim 1, wherein the at least one treatment element comprises at least six treatment elements.

9. The face mask of claim 8, wherein the treatment elements are spaced at a spacing of between 5 and 30 mm.

10. The face mask of claim 1, wherein the electrically insulative lamination layer comprises at least one aperture for the at least one treatment element to be located therein, and/or the electrically insulative lamination layer comprises a material which transmits an emission from the at least one treatment element.

11. The face mask of claim 1, wherein the electrically insulative lamination layer and the flexible and stretchable base substrate comprise a transparent material.

12. The face mask of claim 1, wherein the electrically insulative lamination layer is mounted to the stretchable electronic circuit and/or flexible and stretchable base substrate via an adhesive layer, which optionally comprises a layer of Optically Clear Adhesive.

13. The face mask of claim 12, wherein the adhesive layer comprises at least one aperture for the at least one treatment element to be located therein.

14. The face mask of claim 1, wherein the electrically insulative lamination layer comprises thermoplastic polyurethane.

15. The face mask of claim 1, wherein the electrically insulative lamination layer comprises the same material as the base substrate.

16. The face mask of claim 1, wherein the electrically insulative lamination layer comprises a cured liquid silicone rubber (LSR).

17. The face mask of claim 16, wherein the electrically insulative lamination layer comprises a smooth surface.

18. The face mask of claim 1, further comprising a treatment element protective layer covering the at least one treatment element, preferably wherein the treatment element protective layer comprises a localised silicone drop or a polyurethane acrylate.

19. The face mask of claim 18, wherein the treatment element protective layer comprises a transparent material.

20. The face mask of claim 1, further comprising a strap portion for affixing the face mask to the user, wherein the strap portion is formed of the same material and/or is unitary with the base substrate.

21. The face mask of claim 1, wherein the face mask further comprises a fabric article to which the stretchable base substrate is mounted.

22. The face mask of claim 1, wherein the base substrate comprises a plurality of substrate layers, including at least one layer formed of an optically reflective material.

23. The face mask of claim 1, comprising one or more bores extending through extending through each of the base substrate, electronic circuit, and electrically insulative lamination layer.

24. The face mask of claim 1, wherein the electrically insulative lamination layer comprises an outer cover formed of thermoplastic polyurethane (TPU).

25. A method of manufacturing a face mask for providing therapeutic and/or cosmetic treatment to a user's face, the method comprising:
providing a flexible and stretchable base substrate;
mounting a stretchable electronic circuit directly to the base substrate;
mounting at least one treatment element in communication with the stretchable electronic circuit; and
applying a flexible and stretchable electrically insulative lamination layer over the stretchable electronic circuit.

26. The method of claim 25, further comprising cutting around the face mask to remove it from excess base substrate and form a shape of the face mask.

27. The method of claim 25, wherein the base substrate is mounted on a release paper layer during manufacture, and the method further comprises removing the face mask from the release paper.

28. The method of claim 25, wherein mounting the stretchable electronic circuit directly to the base substrate comprises printing the stretchable electronic circuit on the base substrate using a conductive ink.

29. The method of claim 25, wherein mounting at least one treatment element in communication with the stretchable electronic circuit comprises co-bonding the treatment element to the stretchable electronic circuit.

30. A face mask for providing therapeutic and/or cosmetic treatment to a user's face, the face mask comprising:
a flexible base substrate;
an electronic circuit mounted to the base substrate;
at least one treatment element in communication with the electronic circuit; and
a flexible electrically insulative lamination layer covering the stretchable electronic circuit.

31. The face mask of claim 30, wherein the electrically insulative lamination layer comprises a cured liquid silicone rubber having a smooth surface.
